(19) United States
(12) Patent Application Publication
(10) Pub. No.: US 2021/0244367 A1
(43) Pub. Date: Aug. 12, 2021

(54) METHOD AND SYSTEM FOR DYNAMIC SIGNAL VISUALIZATION OF REAL-TIME SIGNALS

(71) Applicant: NURALOGIX CORPORATION, Toronto (CA)

(72) Inventor: Daniel Raymond MACDONALD, Woodbridge (CA)

(21) Appl. No.: 17/269,400

(22) PCT Filed: Aug. 19, 2019
(86) PCT No.: PCT/CA2019/051126

§ 371 Date: Feb. 18, 2021

Related U.S. Application Data
(60) Provisional application No. 62/725,586, filed on Aug. 31, 2018.

(51) Int. Cl. A61B 5/00; A61B 5/026

(52) U.S. Cl. CPC A61B 5/743; A61B 5/0077; A61B 5/0261; A61B 5/7221

(57) ABSTRACT

A method and system for dynamic signal visualization of real-time signals is provided. The method including: receiving one or more signals from one or more sensors; sampling the one or more signals at a predetermined sampling rate; determining a measure of signal quality for each of the one or more sampled signals; converting each of the measures of signal quality to a displayable visualization; displaying each of the visualizations to a user; updating at least one of the visualizations by determining the measure of signal quality for each newly received sample of the one or more sampled signals; and displaying the updated at least one visualizations to the user.

### TECHNICAL FIELD

The following relates generally to data gathering; and more specifically to a method and system for dynamic signal visualization of real-time signals.

### BACKGROUND

As sensors, such as those located on smartphones, become more prevalent in order to accomplish various automated tasks, capturing good data with such sensors becomes more and more necessary. In an example, certain image processing tasks require performing operations on data captured from a smartphone's one or more cameras. In order for such image processing tasks to operate optimally, the data needs to be sufficiently gathered for the timeframe that the camera is capturing the data.

### SUMMARY

In an aspect, there is provided a computer-implemented method for dynamic signal visualization of real-time signals, comprising: receiving one or more signals from one or more sensors; sampling the one or more signals at a predetermined sampling rate; determining a measure of signal quality for each of the one or more sampled signals; converting each of the measures of signal quality to a displayable visualization; displaying each of the visualizations to a user; updating at least one of the visualizations by determining the measure of signal quality for each newly received sample of the one or more sampled signals; and displaying the updated at least one visualizations to the user.

In a particular case of the method, the one or more signals are sampled at a predetermined sampling rate.

In another case of the method, the measure of signal quality comprises at least one of a signal to noise ratio (SNR), a temporally-averaged signal to noise ratio, and an signal amplitude.

In yet another case of the method, the measure of signal quality is based on an instantaneous sampling of the respective signal.

In yet another case of the method, the visualization comprises one of a histogram, a run chart, and a box plot.

In yet another case of the method, the measure of signal quality is based on an average sampling of the respective signal over a predetermined time period.

In yet another case of the method, the visualization comprises a bar graph or a dial.

In yet another case of the method, the one or more sensors comprise one or more camera sensors capturing a scene, each of the one or more signals represents signals received from a region of the captured scene, and each of the visualizations correspond to the measure of signal quality for the respective region.

In yet another case of the method, the scene comprises a human face and each region comprises a region of the face, and wherein each captured signal represents a blood flow signal for the respective region.

In yet another case of the method, each visualization comprises a histogram.

In another aspect, there is provided a system for dynamic signal visualization of real-time signals, the system comprising one or more processors and a data storage device, the one or more processors configured to execute: a signal module to receive one or more signals from one or more sensors, and sample the one or more signals at a predetermined sampling rate; a quality module to determine a measure of signal quality for each of the one or more sampled signals; and a visualization module to convert each of the measures of signal quality to a displayable visualization, and display each of the visualizations to a user, the visualization module updates at least one of the visualizations from a determination of the measure of signal quality for each newly received sample of the one or more sampled signals and displays the updated at least one visualizations to the user.

In a particular case of the system, the one or more signals are sampled at a predetermined sampling rate.

In another case of the system, the measure of signal quality comprises at least one of a signal to noise ratio (SNR), a temporally-averaged signal to noise ratio, and an signal amplitude.

In yet another case of the system, the measure of signal quality is based on an instantaneous sampling of the respective signal.

In yet another case of the system, the visualization comprises one of a histogram, a run chart, and a box plot.

In yet another case of the system, the measure of signal quality is based on an average sampling of the respective signal over a predetermined time period.

In yet another case of the system, the visualization comprises a bar graph or a dial.

In yet another case of the system, the one or more sensors comprise one or more camera sensors capturing a scene, each of the one or more signals represents signals received from a region of the captured scene, and each of the visualizations correspond to the measure of signal quality for the respective region.

In yet another case of the system, the scene comprises a human face and each region comprises a region of the face, and wherein each captured signal represents a blood flow signal for the respective region.

In yet another case of the system, each visualization comprises a histogram.

These and other aspects are contemplated and described herein. It will be appreciated that the foregoing summary sets out representative aspects of systems and methods to assist skilled readers in understanding the following detailed description.

### BRIEF DESCRIPTION OF THE DRAWINGS

The features of the invention will become more apparent in the following detailed description in which reference is made to the appended drawings wherein:

FIG. 1 shows a system for dynamic signal visualization of real-time signals in accordance with an embodiment;

FIG. 2 shows an exemplary computing environment of the system of FIG. 1;

FIG. 3 shows a method for dynamic signal visualization of real-time signals in accordance with an embodiment;

FIG. 4 illustrates a display generated by the system of FIG. 1 at particular point in time, according to an example case;

FIG. 5 illustrates a display generated by the system of FIG. 1 at particular point in time, according to another example case; and

FIG. 6 illustrates a display generated by the system of FIG. 1 at particular point in time, according to yet another example case.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following relates generally to data gathering; and more specifically to a method and system for dynamic signal visualization for optimal data gathering.

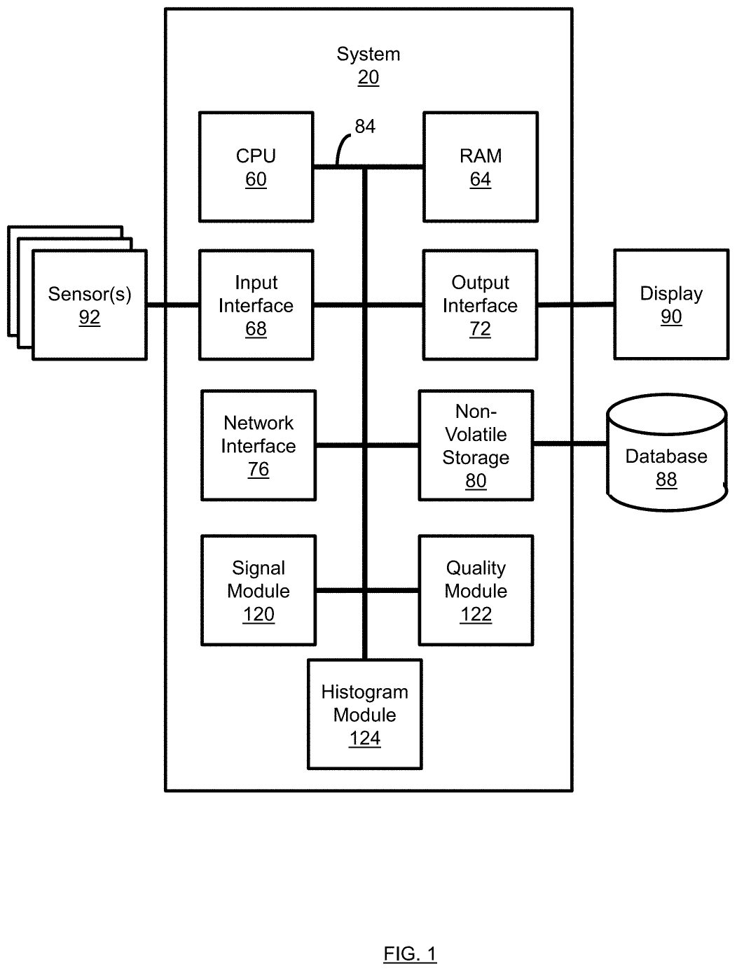

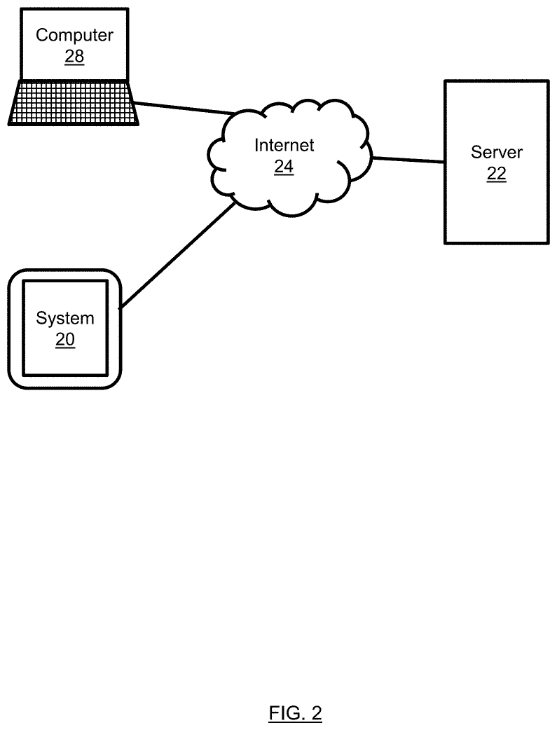

Referring now to FIG. 1, a system 20 for dynamic signal visualization of real-time signals, in accordance with an embodiment, is shown. As shown in FIG. 2, in an exemplary computing environment, the system 20 can be in communication with other computing devices; for example, a computer or a server 22. Other computing devices can also include, for example, tablet computers, smart watches, IOT devices, video cameras, or the like. The system 20 can communicate with the other computing devices locally, or remotely, over a network; for example, the Internet 24.

The system 20 enables visualization to a user of a data gathering process to ensure sufficient and efficient data gathering, though the use of one or more visualizations.

FIG. 1 shows various physical and logical components of the system 20. As shown, the system 20 has a number of physical and logical components, including a central processing unit (“CPU”) 60, random access memory (“RAM”) 64, an input interface 68, an output interface 72, a network interface 76, non-volatile storage 80, and a local bus 84 enabling CPU 60 to communicate with the other components. CPU 60 executes an operating system, a web service, an email server, and a marketing platform. The functionality of the marketing platform is described below in greater detail. RAM 64 provides relatively responsive volatile storage to CPU 60. The input interface 68 enables input to be communicated to the system 20, for example from a user via a keyboard and mouse, or for example via one or more sensors 92 (for example, a camera). The output interface 72 outputs information to output devices, such as a display 90 and/or speakers. The network interface 76 permits communication with other systems, such as computing devices 28, 32. Non-volatile storage 80 stores the operating system and programs, including computer-executable instructions for implementing the web server and the marketing platform, as well as any data used by these services. The non-volatile storage 80 can interface with an internal or external database 88 as a longer-term store of data. During operation of the system 20, the operating system, the programs and the data may be retrieved from the non-volatile storage 80 and placed in RAM 64 to facilitate execution.

In an embodiment, the system 20 is configurable to execute a number of conceptual modules, including a signal module 120, a quality module 122, and a visualization module 124.

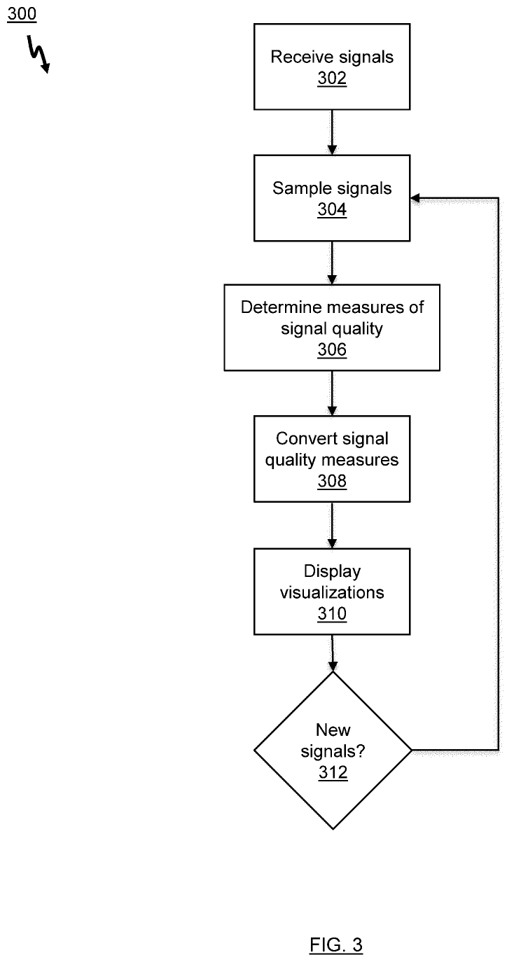

FIG. 3 illustrates a flowchart for a method 300 for dynamic signal visualization of real-time signals, in accordance with an embodiment.

At block 302, the signal module 120 receives one or more signals from one or more sensors 92 in real-time. The sensors 92 can be any sensor that can be visually displayed to a user; for example, a video camera, an infrared camera, a proximity sensor, or the like.

At block 304, the signal module 120 samples the one or more signals at a predetermined rate; for example, where the sensor is a camera, sampling at the camera's frame rate (such as 60 frames-per-second). In further cases, sampling can be performed at any suitable rate, such as based on a moving time window; for example, 0.5, 1, or 10 seconds.

At block 306, the quality module 122 determines a measure of signal quality of each of the one or more sampled signals by performing signal processing on the one or more sampled signals from the one or more sensors 92 in real-time. The signal quality can be determined, for example, by determining a signal to noise ratio (SNR) for each of the signals, a temporally-averaged signal to noise ratio for the signals, determining amplitudes of the signals, or the like. In an example, the quality of the signals, having been digitally converted using a 16-bit conversion, is a signal to noise ratio represented as the number of low-order bits of random noise. The SNR can be estimated, for example, using a ratio of a summed squared magnitude to that of the noise, using a modified periodogram, or the like. In some cases, the measure of signal quality can be based on an instantaneous sampling of the respective signal. In further cases, the measure of signal quality can be based on an average sampling of the respective signal over a predetermined time period (for example, 500 milliseconds).

At block 308, the visualization module 124 converts each of the measures of signal quality to a displayable visualization. In some cases, the measures of signal quality can be converted to a temporal visual representation showing the measures of signal quality over time; for example, a histogram, a run chart, a box plot, or the like. In other cases, the measures of signal quality can be converted to a static visual representation showing the measure of signal quality at the approximate current instance; for example, a bar graph, a dial, or the like. In most cases, the visualization module 124 can update the visualizations as new signal quality measures are received.

At block 310, the visualization module 124 displays each of the visualizations to a user with the display 90 via the output interface 72. In a particular case, each of the visualizations are shown in association with the respective received signal from the respective sensor 92.

At block 312, if new signals are received, the system 20 updates the visualizations by repeating blocks 304 to 310 for the new signals; thus allowing the visualizations to be updated in real-time.

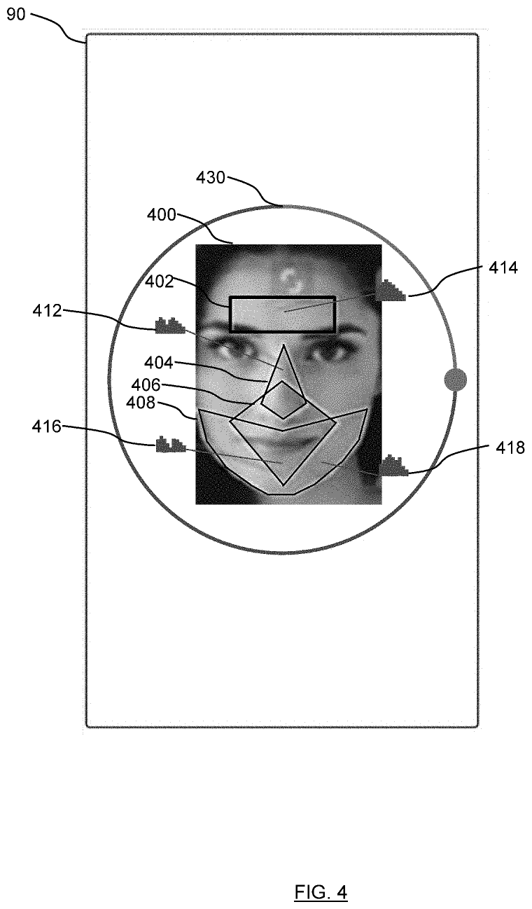

FIGS. 4 to 6 show illustrative and non-limiting examples of applications of the system 20 as outputted on the display 90.

FIG. 4 illustrates the display showing the system 20 at an exemplary time point. In this example, the system 20 is receiving signals from a camera sensor 92. The camera sensor is directed at a user's face 400. Each signal is received from a region of interest (ROI), a first region 402, a second region 404, a third region 406, and a fourth region 408, each associated with a region of the user's face 400. As shown in FIG. 4, the visualization module 124 may cause the display 90 to overlay bounding boxes upon the subject (the user's face in this example) around each ROI, providing the user with context as to the portion of the image for which a specific signal quality measure relates.

The signal from each ROI 402, 404, 406, and 408 is sampled and converted into a visualization. In this example, the first region 402 is converted into the first visualization 412, the second region 404 is converted into the second visualization 414, the third region 406 is converted into the third visualization 416, and the fourth region 408 is converted into the fourth visualization 418. In this example, the visualizations 412, 414, 416, and 418 are histograms representing signal quality over time. In this example, there is also derivative visualization 430 displayed by the visualization module 124 showing another property of the signals. In an example, the derivative visualization 430 can represent an average signal quality. In another example, the derivative visualization 430 can represent the time remaining to have enough data to proceed with an operation on the signal data at the current signal quality acquisition.

In this way, the signals for each region can be sampled every frame and the system 20 can provide an immediate visual user feedback on signal quality. Where the sensor 92 is a camera, the histogram visualizations that are either under or over saturated may result in a weaker signal extraction due to signal clipping, so the histogram visualization can advantageously notify and help the user adjust their position and/or lighting for better signal acquisition.

In an example, the example of FIG. 4 can be used to determine blood flow data in the ROIs. The histograms visualization, which can have multiple dominate peaks, can thus be an indication that the user is being lit by multiple light sources. This can result in multiple blood flow signals present at different signal offsets within the sampled region and can reduce signal quality (particularly, when averaged). In this example, when visualizing the histogram peak movements across frames, it will likely shimmer horizontally across a few histogram bins as the facial color changes due to the blood flowing in and out of the regions. The amplitude of the histogram bins may also move up and down over time as more signals are sampled at the same or similar color as the blood flows in and out of a region. If the amplitude is varying too much between frames, it can be indicative of region shape (sample area size changing) due to participant movement and will likely result in poorer signal extraction (area averaging changes).

Thus, having multiple visualizations, sampled from different regions of the face, is advantageous because different regions are illuminated differently due to positioning relative to a light source, and may have stronger or weaker signals accordingly. Thus, presenting visualizations, and especially multiple visualizations, can provide the user with feedback to tune the signal acquisition in order to increase the quality of the signals. For example, tuning can include moving or directing the camera for better signal acquisition or changing the lighting for better signal acquisition.

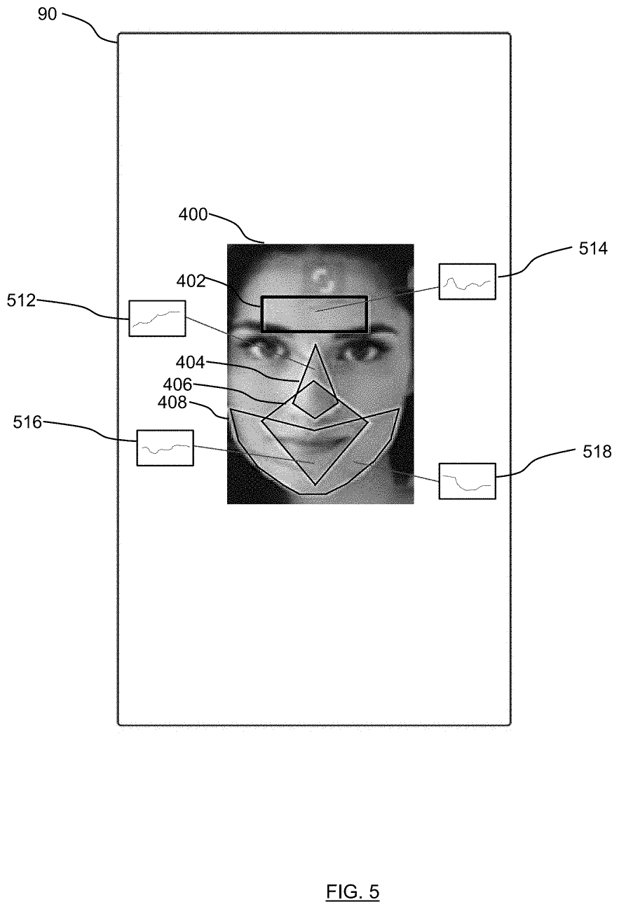

FIG. 5 illustrates an example similar to that of FIG. 4. In this example, the first region 402 is converted into the first visualization 512, the second region 404 is converted into the second visualization 514, the third region 406 is converted into the third visualization 516, and the fourth region 408 is converted into the fourth visualization 518. In this example, the visualizations 512, 514, 516, and 518 are line plots representing signal quality over time.

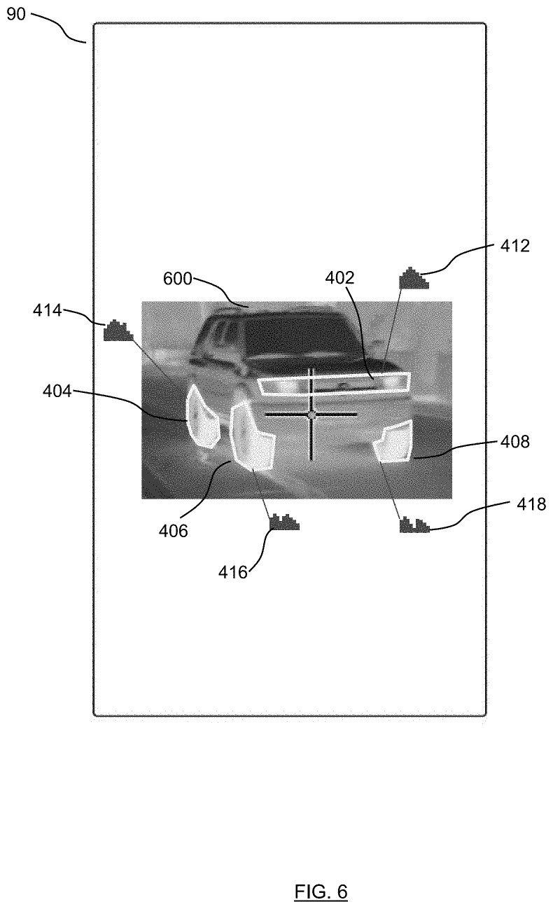

FIG. 6 illustrates an example display 90 showing the system 20 at an exemplary time point. In this example, the system 20 is receiving signals from an infrared camera sensor 92. The infrared camera sensor is directed at an object, in this case a vehicle 600. Each signal is received from a thermal region of interest (ROI), a first region 602, a second region 604, a third region 606, and a fourth region 608, each associated with a region of the vehicle 600. The signal from each ROI 602, 604, 606, and 608 is sampled and converted into a visualization. In this example, the first region 602 is converted into the first visualization 612, the second region 604 is converted into the second visualization 614, the third region 606 is converted into the third visualization 616, and the fourth region 608 is converted into the fourth visualization 618. In this example, the visualizations 612, 614, 616, and 618 are histograms representing signal quality over time. In this example, the system 20 can use the infrared image for machine image recognition. The visualizations 612, 614, 616, and 618 can thus be used to display to the user if the regions are being sufficiently sampled with a high enough quality of signal in order to properly perform the image recognition task. Advantageously, the user can move, position, and direct the infrared camera sensor based on the visualizations 612, 614, 616, and 618 for higher quality signal acquisition; potentially resulting in a quicker recognition.

While four regions of interest were shown in the preceding examples, any number of regions can be used. In further cases, other divisions of signals can be used; for example, where there are multiple sensors directed at a subject, each sensor can provide its own signal. In this case, each of the sensor outputs and its associated signal quality can be displayed to the user on the display 90.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

1. A computer-implemented method for dynamic signal visualization of real-time signals, comprising:
receiving one or more signals from one or more sensors;
sampling the one or more signals at a predetermined sampling rate;
determining a measure of signal quality for each of the one or more sampled signals;
converting each of the measures of signal quality to a displayable visualization;
displaying each of the visualizations to a user;
updating at least one of the visualizations by determining the measure of signal quality for each newly received sample of the one or more sampled signals; and
displaying the updated at least one visualizations to the user.

2. The method of claim 1, wherein the one or more signals are sampled at a predetermined sampling rate.

3. The method of claim 1, wherein the measure of signal quality comprises at least one of a signal to noise ratio (SNR), a temporally-averaged signal to noise ratio, and an signal amplitude.

4. The method of claim 3, wherein the measure of signal quality is based on an instantaneous sampling of the respective signal.

5. The method of claim 4, wherein the visualization comprises one of a histogram, a run chart, and a box plot.

6. The method of claim 3, wherein the measure of signal quality is based on an average sampling of the respective signal over a predetermined time period.

7. The method of claim 6, wherein the visualization comprises a bar graph or a dial.

8. The method of claim 1, wherein the one or more sensors comprise one or more camera sensors capturing a scene, each of the one or more signals represents signals received from a region of the captured scene, and each of the visualizations correspond to the measure of signal quality for the respective region.

9. The method of claim 8, wherein the scene comprises a human face and each region comprises a region of the face, and wherein each captured signal represents a blood flow signal for the respective region.

10. The method of claim 9, wherein each visualization comprises a histogram.

11. A system for dynamic signal visualization of real-time signals, the system comprising one or more processors and a data storage device, the one or more processors configured to execute:
a signal module to receive one or more signals from one or more sensors, and sample the one or more signals at a predetermined sampling rate;
a quality module to determine a measure of signal quality for each of the one or more sampled signals; and
a visualization module to convert each of the measures of signal quality to a displayable visualization, and display each of the visualizations to a user, the visualization module updates at least one of the visualizations from a determination of the measure of signal quality for each newly received sample of the one or more sampled signals and displays the updated at least one visualizations to the user.

12. The system of claim 11, wherein the one or more signals are sampled at a predetermined sampling rate.

13. The system of claim 11, wherein the measure of signal quality comprises at least one of a signal to noise ratio (SNR), a temporally-averaged signal to noise ratio, and an signal amplitude.

14. The system of claim 13, wherein the measure of signal quality is based on an instantaneous sampling of the respective signal.

15. The system of claim 14, wherein the visualization comprises one of a histogram, a run chart, and a box plot.

16. The system of claim 13, wherein the measure of signal quality is based on an average sampling of the respective signal over a predetermined time period.

17. The system of claim 16, wherein the visualization comprises a bar graph or a dial.

18. The system of claim 11, wherein the one or more sensors comprise one or more camera sensors capturing a scene, each of the one or more signals represents signals received from a region of the captured scene, and each of the visualizations correspond to the measure of signal quality for the respective region.

19. The system of claim 18, wherein the scene comprises a human face and each region comprises a region of the face, and wherein each captured signal represents a blood flow signal for the respective region.

20. The system of claim 19, wherein each visualization comprises a histogram.
